(19) United States
(12) Patent Application Publication
(10) Pub. No.: US 2008/0076831 A1
(43) Pub. Date: Mar. 27, 2008

(54) Hemorrhoid reliever and method of use

(76) Inventor: Bernard Goetz, New York, NY (US)

Correspondence Address: Alan Chan, Suite 700, 225 Broadway, New York, NY, 10007, US

(21) Appl. No.: 11/525,357

(22) Filed: Sep. 22, 2006

(51) Int. Cl. A61K 31/155

(52) U.S. Cl. 514/637

(57) ABSTRACT

A composition containing chlorhexidine enabling treatment of many types of discomforts, including hemorrhoid pain and itching without discomfort.

### BACKGROUND OF THE INVENTION

In one aspect, this invention relates to a composition of matter useful for treating bodily discomforts. In another aspect, this invention relates to a method for treating bodily discomforts.

Several topical agents (creams, ointments, liniments and the like) have been utilized for the relief of hemorrhoids. Most of these have provided a little, but only temporary, relief to persons suffering from pain. The burning side effect has also discouraged the use of antiseptics to treat other types of discomfort, such as itching.

A chlorhexidine based pain reliever which does not irritate the skin or cause a burning discomfort, would be extremely desirable and acceptable to patients and people in general who are experiencing the types of pain or discomfort associated with hemorrhoids.

### OBJECTS OF THE INVENTION

It is an object of this invention to provide a chlorhexidine based pain reliever that does not burn when applied topically.

It is another object of this invention to provide a method for formulating a no-burn chlorhexidine-based pain reliever that relieves pain and discomfort and in which the chlorhexidine is fully functional, one that provides disinfectant properties.

It is a further object of this invention to provide a method for treating pain and discomfort with chlorhexidine that does not burn the skin when applied topically. The preferred treatment is for wounds and hemorrhoids.

### SUMMARY OF THE INVENTION

In accordance with one aspect of the invention, there is provided a composition comprising a carrier, chlorhexidine, and an encapsulation agent.

In accordance with another aspect of the invention, there is provided a method for treating a victim of pain or discomfort. The treatment comprises applying the above described composition topically to the skin of the person near an area affected by the pain or discomfort.

In accordance with a further aspect of the invention, there is provided a method for making a composition useful for topical application to treat pain or discomfort. The method is carried out by mixing a carrier to form an aqueous solution, and optionally adding an encapsulation agent to enhance absorption into the skin. The resulting aqueous solution preferably can have a cream-like viscosity.

### DETAILED DESCRIPTION OF THE PREFERRED EMBODIMENTS

The composition of the invention comprises chlorhexidine as a active ingredient and at least one inactive ingredient. The ingredients are contained in a carrier fluid. Chlorhexidine works by killing the bacteria. As a substitute for chlorhexidine, chlorhexadine may also be used.

Generally speaking, the composition will contain in the range up to 2% by weight of chlorhexidine. Compositions containing more than 2% by weight of chlorhexidine will also provide a therapeutic effect, up to 99% by weight chlorhexidine. Compositions containing 0.025% to 20% by weight of chlorhexidine could be used. Compositions of 0.025 to 2% by weight are considered usable as well. Even so, compositions containing in the range of 0.025% to 0.2% by weight of chlorhexidine are preferred

In the present invention, chlorhexidine is mixed with a carrier fluid. Preferably, the carrier fluid is water-based and forms an aqueous solution containing the ingredients. However, the carrier may be a fluid such as an oil based carrier, a fat based carrier, a fatty alcohol based carrier or combination of these.

It is possible to add more than one histidine to achieve the analgesic reaction. Specifically, amine and caine type local anethestics, such as benzocaine and lidocaine, act differently as anesthetics not producing an analgesic effect which is achieved by adding an additional irritant, such as a histamine hydrochloride or most preferably a histamine dihydrochloride.

Any one of the following histidines, or combinations thereof, are considered usable in this invention, L-histidines, histamine dihydrocholoride, DL-histidine, D-histidine hydrochloride monohydrate, L-histidine hydrochloride monohydrate, L-histidine methyl ester dihydrochloride, L-histidinol dihydrochloride.

When histidines are used in the invention, it is preferred that the weight percent of the material is between 0.001% and about 1.0 wt. %.

Other histidines are considered usable within the scope of this invention, as well as the second irritant. It is possible though, that the invention will work well with out the second irritant.

The novel composition further comprises an encapsulation agent such as colloidal oatmeal, hydrogenated lecithin, dipotassium glycyrrhizinate or other similar encapsulation agents, or even combinations of these agents with the chlorhexidine and second irritant. Typically, 0.35 wt. % colloidal oatmeal is used in this invention, although any amount between 0.20 and up to about 0.60 wt. Percent can be used. The colloidal oatmeal works within the scope of this invention because it contains hydrophilic colloids. These colloids help to provide a protective barrier on the skin to control inflammation. In addition, histidines, such as L-histidines, are present in colloid oatmeal. Histidines can be present in the oats in weight percents up to 3% of the total amino acids in the oats.

Hydrogenated lecithin is an emulsifier and stabilizer for solutions. In addition, it is used to reduce inflammation on the skin. Additionally, the lecithin is used to reduce irritation that differs from inflammation. Inflamed skin is red and hot, irritated skin is itchy without necessarily being inflamed and red.

Esters of amino acids maybe added to the formulation. Esters of amino acid usable in the scope of this invention are preferably menthyl and lauryl esters of amino acids. The active element in this component is menthol that acts as an analgesic. It is considered within the scope of the present invention to use enough menthyl lauryl pidolate to attain between 0.1 and 16 wt % menthol in the formulation.

It is the non-burning disinfectant effect which make this invention unique.

Additional components can be added to the formulation, such as “lavender flower oil” or lavender oil, and a “bergaptene-free” bergamot oil or bergamot extract is expected to be beneficial in that the lavender provides a muscle relaxant characteristic.

Also, for enhancing the composition, other elements may be added to the composition. The following are contemplated: 1. Arnica montana 2. Hypericum perforatum (known as St. John's Wort) 3. Aloe barbadensis gel 4. Citric acid to adjust the pH of the compound 5. Propylene glycol with methyl and propyl parabens as preservatives 6. A chealating agent to keep the product from separating, such as edetatedisodium 7. Triethanolamine hydrochloride which acts as a reagent 8. Other preservatives

Still others considered usable in the present invention are phenoxy ethanol, ethyl paraben, and butyl paraben as preservatives, or in the preservative system. Other ingredients such as inositol, methyl paraben, propyl paraben, hydroxy ethyl cellulose can be used therein, for formulations which are gels rather than creams. Carbomer 940 can be used to make the formula into a gel rather than a cream.

Xanthum gum can be added to the invention to provide a higher density compound, and act as a thickening agent. Other elements, such as licorice extract, glycerial polymethacrytate and hydroxypropyl cellulose can be used in various formulations of the basic invention.

A suspension agent can be added to the formula of the present invention. Alkyl benzoate is considered usable within the scope of the present invention.

Deionized water is an excellent aqueous carrier for the present invention.

In the method of the invention, a victim of pain or discomfort is treated by applying the above-described composition topically to the skin of the victim near an area affected by the pain or discomfort. The types of pain or discomfort to which the invention may be applied include those discussed in the background of the invention. Generally speaking, the inventive composition, preferably in liquid form, is applied to the selected area, such as a joint, and rubbed in. The amount applied is not critical. Generally, it should be applied in an amount that is sufficient to wet the area of application.

For the treatment of pruritus or itching, the application of the composition can be repeated as required to control the discomfort. When the preferred composition of the invention is applied, it provides significant relief from the itching caused by hemorrhoids, without a burning sensation. The relief lasts for several hours. It is surprising that a chlorhexidine based composition would be useful for the treatment of such discomfort. To enhance the antipruritic effect, additional compounds can be added to the formulation. These components can be methyl sulphonyl methane, sodium bicarbonate, calamine, allatoin, kaolin, and combinations thereof.

It is contemplated to be within the scope of the present invention to use this formulation for a gel, a cream, an opaque cream, a spray using propellants, such as butyl propellants, and a liquid or lotion, such as a roll on.

It is even contemplated that the present invention could be used as a patch for treatment as well. Propellant for the spray on composition contemplated as usable herein can be selected from the group butane, propane, isobutane, and combinations thereof. A foam version of the formulation, additionally using a propellant and a surfactant is considered within the scope of the present invention. A preferred surfactant is a member of the group of amine oxides. The most preferred surfactant is alkyl dimethyl amine oxide.

In the preferred embodiment, the viscosity adjusting agent is a member of the group comprising magnesium chloride, citric acid, sodium chloride, and combinations thereof, and the analgesic agent is selected from the group histamine dihydrochloride, glucasomine, white willow bark, ibuprofen, salicylamide, salicyclic acid and salsalate and combinations thereof. The encapsulation agent is preferably selected from the group consisting of xanthan gum, gellan gum, arabica gum, anacia gum, gum tragacanth, guar gum, dammar resin, elemi resin, sandarac resin, polyvinyl acetate, polyester, amide, carboxymethyl cellulose, carboxymethyl hydroxyethyl cellulose, carboxypolymethylene and combinations thereof.

Additionally, the composition can further comprise a skin and tissue emollient. For example, panthenol, dexpanthenol, vitamin B complex factor, glycerin, glycerol, sodium hyaluronate, myristal myristate, propylene glycol, natural nut oils and combinations thereof. If an amide is used, it is preferrably powdered nylon or powdered sulfonamide.

The most preferred topical carrier is selected from the group comprising: aqueous carriers, oil based carriers, fat based carriers, and fatty alcohol based carriers, water or combinations thereof.

The preferred ester is an alkyl ester.

For the preferred embodiment, the analgesic agent is a member of the group comprising histamine hydrochloride and methylnicotinate.

The preferred histamine is histamine hydrochloride is a dihydrochloride.

The analgesic agent has a weight percent in the range of about 0.025% up to about 0.1%.

The capsicum extract is in the range of 0.01 to 20.0% by weight.

The composition can additionally comprise a light scattering element selected from the group: titanium dioxide, zinc oxide, and benzophenones, methoxy cinnamate, para amino benzoic acid, octyl, dodecyl, neopentanoate, aluminum stearate with titanium dioxide, aluminum oxide with titanium dioxide, and combinations thereof.

The invention can also comprise an anti-itch agent which is a member of the group: methyl sulphonyl methane, sodium bicarbonate, calamine, allantoin, kaolin, and combinations thereof.

The invention relates to a hemorrhoid cream comprising an effective amount to treat hemorrhoids comprising: a topical carrier; a transdermal component selected from the group comprising, ester, and combinations thereof; a chlorhexidine ; an encapsulation agent selected from the group comprising gums and resins and their derivatives; a solubility agent; a viscosity adjusting agent; and an analgesic agent.

The invention relates to a lotion comprising: a topical carrier; a transdermal component selected from the group comprising ester, methylsulfonylmethane, benzyl alcohol, benzoic acid and combinations thereof; a chlorhexidine; an encapsulation agent selected from the group comprising gums and resins and their derivatives; a solubility agent; a viscosity adjusting agent; an analgesic agent.; and an emulsifying agent. The emulsifying agent is preferably glyceryl monostearate and polysorbate.

The invention also relates to a spray on formulation comprising: a propellant; a transdermal component selected from the group comprising, ester, methylsulfonylmethane, benzyl alcohol, benzoic acid and combinations thereof; a capsicum extract; an encapsulation agent selected from the group comprising gums and resins and their derivatives; a solubility agent; a viscosity adjusting agent; and an analgesic agent.

The propellant can be a butane, propane, isobutane, and combinations thereof.

The invention also pertains to a foam formulation comprising: a surfactant comprising an amine oxides; a propellant; a transdermal component selected from the group comprising ester, methylsulfonylmethane, benzyl alcohol, benzoic acid and combinations thereof; a chlorhexidine; an encapsulation agent selected from the group comprising gums and resins and their derivatives; a solubility agent; a viscosity adjusting agent; and an analgesic agent. The most preferred surfactant is amine oxide is alkyl dimethyl amine oxide.

The inventive method also works for victim that suffer from the discomfort caused by hemorrhoids.

While the above invention has been described with reference to certain preferred embodiments, the scope of the present invention is not limited to these embodiments. One skilled in the art may find variations of these preferred embodiments which, nevertheless, fall within the spirit of the present invention, whose scope is defined by the claims set forth below.

1-13. (canceled)

14. A method for treating at least one of pain and discomfort suffered by an individual afflicted with hemorrhoids, said method comprising applying to a hemorrhoid afflicting an individual in need of such treatment a therapeutically effective amount of a composition comprising, as an active ingredient, at least one of chlorhexidine and chlorhexadine, and a topical carrier, said active ingredient being present in the composition in an amount of up to about 99% by weight of said composition.

15. The method according to claim 14, wherein said active ingredient is present in said composition in an amount of between about 0.025% and 20% by weight of said composition.

16. The method according to claim 14, wherein said active ingredient is present in said composition in an amount of between about 0.025% and 0.2% by weight of said composition.

17. The method according to claim 14, wherein the topical carrier is a fluid and wherein said fluid is selected from the group consisting of water-based fluids which form an aqueous solution with the active ingredient, oil-based carrier fluids, fat-based carrier fluids, fatty alcohol-based carrier fluids and combinations thereof.

18. The method according to claim 14, wherein said composition additionally comprises at least one analgesic agent selected from the group consisting of histamine hydrochloride, methylnicotinate, histidine glucasomine, white willow bark, ibuprofen, salicylamide, salicyclic acid, salsalate and combinations thereof.

19. The method according to claim 18, wherein the analgesic agent is present in the composition in an amount of between about 0.025% and 0.1 wt. % of said composition.

20. The method according to claim 19, wherein said analgesic agent is a histidine and said histidine is selected from the group consisting of L-histidine, histamine hydrochloride, histamine dihydrochloride, DL-histidine, D-histidine hydrochloride monohydrate, L-histidine hydrochloride monohydrate, L-histidine methyl ester dihydrochloride, L-histidinol dihydrochloride and combinations thereof

21. The method according to claim 14, wherein the composition additionally comprises an encapsulation agent present in the composition in an amount of between about 0.20 and 0.60 wt. % of said composition.

22. The method according to claim 21, wherein the encapsulation agent is selected from the group consisting of colloidal oatmeal, hydrogenated lecithin, dipotassium glycyrrhizinate, xanthan gum, gellan gum, arabica gum, anacia gum, gum tragacanth, guar gum, dammar resin, elemi resin, sandarac resin, polyvinyl acetate, polyester, amide, carboxymethyl cellulose, carboxymethyl hydroxyethyl cellulose, carboxypolymethylene and combinations thereof.

23. The method according to claim 14, wherein the composition additionally comprises an ester of an amino acid.

24. The method according to claim 23, wherein the amino acid ester is selected from the group consisting of methyl and lauryl esters.

25. The method according to claim 24, wherein the composition comprises a sufficient amount of methyl lauryl pidolate to add between about 0.1 and 16 wt. % of menthol to the composition.

26. The method according to claim 14, wherein the composition additionally comprises a skin and tissue emollient.

27. The method according to claim 26, wherein the skin and tissue emollient is selected from the group consisting of panthenol, dexpanthenol, vitamin B complex factor, glycerin, glycerol, sodium hyaluronate, myristal myristate, propylene glycol, natural nut oils and combinations thereof.

28. The method according to claim 14, wherein the composition additionally comprises an anti-itch agent and wherein said agent is selected from the group consisting of methyl sulphonyl methane, sodium bicarbonate, calamine, allantoin, kaolin and combinations thereof.

29. A method for treating at least one of pain and discomfort suffered by an individual afflicted with hemorrhoids, said method comprising applying to a hemorrhoid afflicting an individual in need of such treatment a therapeutically effective amount of a gel composition adapted for treating said hemorrhoid, said gel composition comprising an active component selected from the group consisting of chlorhexidine, chlorhexadine and a combination thereof, said active component present in the composition in an amount of up to about 99% by weight of the composition, a topical carrier and an encapsulation agent.

30. The method according to claim 29, wherein the gel composition additionally comprises at least one additional component selected from the group consisting of a solubility agent, a viscosity adjusting agent and an analgesic agent.

31. A method for treating at least one of pain and discomfort suffered by an individual afflicted with hemorrhoids, said method comprising applying to a hemorrhoid afflicting an individual in need of such treatment a therapeutically effective amount of a cream composition adapted for treating said hemorrhoid, said cream composition comprising an active component selected from the group consisting of chlorhexidine, chlorhexadine and a combination thereof, said active component present in the composition in an amount of up to about 99% by weight of the composition, a topical carrier and an encapsulation agent.

32. The method according to claim 31, wherein the cream composition additionally comprises at least one additional component selected from the group consisting of a solubility agent, a viscosity adjusting agent and an analgesic agent.

33. A method for treating at least one of pain and discomfort suffered by an individual afflicted with hemorrhoids, said method comprising applying to a hemorrhoid afflicting an individual in need of such treatment a therapeutically effective amount of a lotion composition adapted for treating said hemorrhoid, said lotion composition comprising an active component selected from the group consisting of chlorhexidine, chlorhexadine and a combination thereof, said active component present in the composition in an amount of up to about 99% by weight of the composition, a topical carrier and an encapsulation agent.

34. The method according to claim 33, wherein the lotion composition additionally comprises at least one additional component selected from the group consisting of a solubility agent, a viscosity adjusting agent and an analgesic agent.
